(19) United States
(12) Patent Application Publication
(10) Pub. No.: US 2024/0277516 A1
(43) Pub. Date: Aug. 22, 2024

(54) Hydrodynamically Actuated Preservative Free Dispensing System Having a Collapsible Liquid Reservoir

(71) Applicant: Bausch + Lomb Ireland Limited, Dublin (IE)

(72) Inventor: Yehuda Ivri, Newport Coast, CA (US)

(73) Assignee: Bausch + Lomb Ireland Limited, Dublin (IE)

(21) Appl. No.: 18/653,727

(22) Filed: May 2, 2024

Related U.S. Application Data
(63) Continuation of application No. 17/233,135, filed on Apr. 16, 2021.
(60) Provisional application No. 63/011,808, filed on Apr. 17, 2020.
(60) Provisional application No. 63/166,754, filed on Mar. 26, 2021.

(51) Int. Cl. A61F 9/00; A61J 1/14

(52) U.S. Cl. CPC A61F 9/0008; A61J 1/1443

(57) ABSTRACT

Multi-dose preservative-free ocular fluid delivery devices are provided. The fluid delivery device includes a fluid dispensing system and a fluid package for storing a liquid therein and supplying said liquid to the dispensing system. The dispensing system comprises an elongated chamber which includes a check valve which defines a frontal closure to the chamber. The valve is normally closed and hermetically seals the chamber. The dispenser includes a vibration motor that induces oscillations to the chamber and to the fluid within. The oscillations of the chamber impart momentum to the fluid stored in the chamber which in turn imparts force that cyclically opens the valve to dispense streams or liquid droplets. Fluid is dispensed only when the motor oscillates while otherwise the valve is hermetically closed. The fluid package can be made collapsible to remove the need to vent it.

### CROSS REFERENCE TO RELATED APPLICATIONS

This application is a continuation patent application of U.S. application Ser. No. 17/233,135, filed Apr. 16, 2021, which claims the benefit of and priority to U.S. Provisional Patent Application No. 63/011,808, filed Apr. 17, 2020, and U.S. Provisional Patent Application No. 63/166,754, filed Mar. 26, 2021, each of which are incorporated herein by reference in their entireties.

### FIELD OF THE INVENTION

The present invention generally pertains to devices for dispensing fluid medicines and more particularly pertains to such devices that store and deliver ophthalmic preservative-free medicines, specifically configured to increase the ease of use and enhance patient compliance with dosing instructions for the medicine.

### BACKGROUND

Ease of dispensing fluid medicines and compliance with dosing instructions are primary concerns with all patients. In particular, preservative-free dispensing bottles such as ophthalmic squeeze dispensers typically require greater actuation force due to a valve mechanism that seals the dispensing nozzle to prevent bacterial ingress and contamination. Such system requires much higher pressure to operate and hence much higher squeeze force is required. In addition, prior art dispensing bottles dispense only in an upside-down orientation which require an inconvenient head maneuver and which together with higher actuation force further increases the inconvenience.

Dispensers of the kind in question are known from the prior art, for example from U.S. Pat. Nos. 6,095,376, 9,676,525, US 2014/0336596, US 2016/0107180, U.S. Pat. Nos. 9,238,532, 8,056,766, 8,863,998, and 10,105,720. The dispenser shown in US 2014/0336596 comprises an outlet channel which connects the liquid reservoir to the outlet opening through an outlet valve which is arranged in the outlet channel and which opens when the bottle is squeezed and pressure is generated. Such preservative-free squeeze bottles typically require about 25-28N of squeeze force (Ophthalmic Squeeze Dispenser-Drug Development and Delivery October 2017 Vol. 17 No. 7 page 40). Elderly patients, or other patients lacking enough strength and/or dexterity in their hands, often experience problems dispensing medicine from such bottles. This work provides preservative free ocular dispensing device that can be held horizontally, or in any convenient orientation while the actuation is done effortlessly by an electrical switch. This provides a cost effective solution that is consistent with standard drug packaging processes. The fluid reservoir can be made collapsible to remove the need to vent it.

### SUMMARY

Multi-dose preservative-free ocular fluid delivery devices are provided. The fluid delivery device includes a fluid dispensing system and a fluid package for storing a liquid therein and supplying said liquid to the dispensing system. The dispensing system comprises an elongated chamber which includes a check valve which defines a frontal closure to the chamber. The valve is normally closed and hermetically seals the chamber. In this work the chamber includes a vibration motor that induces oscillations to the chamber and to the fluid within. The oscillations of the chamber impart momentum to the fluid stored in the chamber which in turn imparts force that cyclically opens the valve to dispense streams or liquid droplets. Fluid is dispensed only when the motor oscillates while otherwise the valve is hermetically closed.

The check valve can include a flexible plate which includes a conical aperture that extends through its thickness, the valve can further include a stationary spherical member that engages tangentially with the inner wall of the conical aperture to create a hermetic sealed closure. The plate can be made of elastomer that has a modulus of elasticity ranging between 0.1-1.2 GPa. The circumference of the plate can be attached to the chamber by a retaining ring that engages with the chamber in an interference fit to create the hermetically sealed closure.

The conical aperture extends through the thickness of the plate such that droplets are dispensed through the smaller opening of the aperture while the larger side of the aperture is in fluid communication with the chamber.

The spherical member may include an antibacterial coating which covers the area of the spherical member that is between the tangential engagement line and the small opening of the aperture.

The vibrational motor oscillates the chamber and the fluid within the chamber. Consequently, cycles of hydrodynamic pulses are generated causing the valve to cyclically open and dispense fluid. Here this phenomenon is characterized by oscillatory interactions between the valve and the surrounding fluid. The hydrodynamic force generated by the momentum. of the fluid opens the valve and allows fluid flow through the aperture.

Fluid is dispensed only when the hydrodynamic force is sufficiently high to deform the aperture while otherwise the aperture hermetically seals the chamber. The system prevents ingress of microorganism into the chamber allowing storage of preservative free pharmaceutical. This work provides an electrically operated preservative-free dispensing system that is convenient and cost effective.

### BRIEF DESCRIPTION OF THE DRAWINGS

FIG. 1A is a cross sectional view of an embodiment of the invention.

FIG. 1B is a detail cross sectional view of the embodiment of FIG. 1A.

FIG. 1C is a 3D view of the embodiment of FIGS. 1A-B.

FIG. 2A is a side view and partial cross section view of an embodiment of the invention in operation.

FIG. 2B is a detail cross section view of the example of FIG. 2A.

FIG. 3 illustrates a perspective view of a vibration motor as used in embodiments of the invention.

FIG. 4A is a frontal view of an embodiment of the invention.

FIG. 4B is a side view of the embodiment of FIG. 4A.

FIG. 4C is a detail cross sectional view of the embodiment of FIG. 4A.

FIG. 5A is a frontal view of an exemplary device enclosed in a housing.

FIG. 5B is a side view of the example of FIG. 5A.

FIGS. 6A-B are perspective views of a device having a housing that includes a swivel cover.

FIGS. 7A-B shown another embodiment of the invention.

FIGS. 8A-B are cross section views of the example of FIGS. 7A-B.

### DETAILED DESCRIPTION

This work describes dispensing devices and methods for delivery of preservative-free solutions or suspensions for ocular administration of ophthalmic drugs. The dispensing devices include a droplet ejecting system that is fluidly connected to an ampoule package containing a liquid to be dispensed. The droplet ejecting system includes a chamber having a check valve that defines a front closure to the chamber. The dispensing system further includes a vibration motor that oscillates the chamber and induces hydrodynamic pulses which consequently causes the valve to cyclically open and eject fluid droplets. The valve is normally closed and hermetically sealing the chamber. The valve opens exclusively in response to hydrodynamic pulses induced by the oscillation of the chamber. In this way fluid is dispensed only when the device is actuated while otherwise the aperture hermetically seals the device and prevents ingress of bacterial and microorganisms thereby allowing storage of preservative-free pharmaceutical formulation. The use of a vibration motor further enables convenient and cost effective, electronically controlled administration.

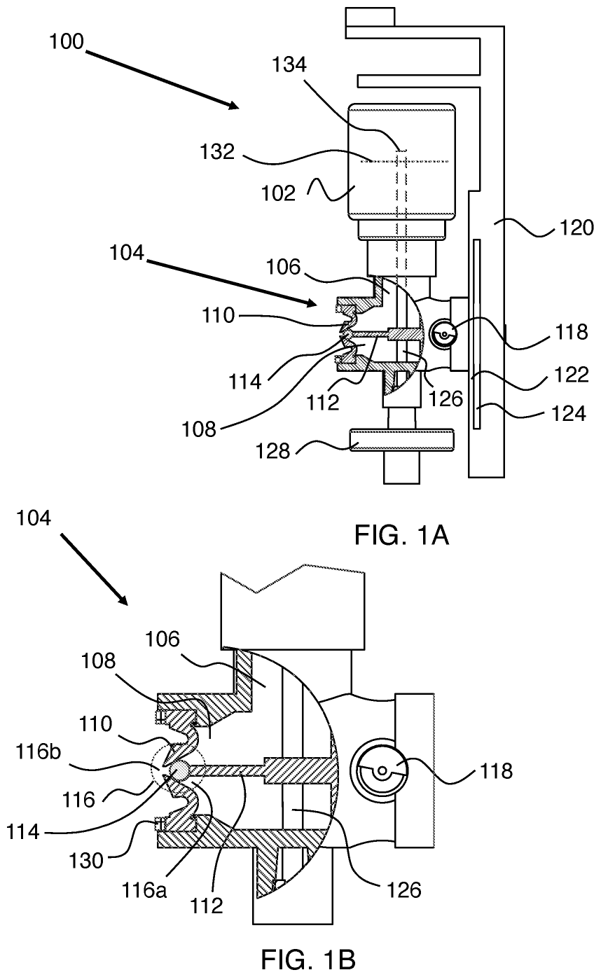

FIG. 1A and FIG. 1B illustrate a side view and an enlarged partial section view, respectively, of a fluid delivery device 100. Delivery device 100 includes a fluid reservoir 102 and dispensing system 104 connected to each other in fluid transmission relationship though passage 106. Dispensing system 104 includes a fluid chamber 108 and also includes a check valve including aperture plate 110 which provides frontal closure to the chamber 108. Referring to FIG. 1B it can be seen that aperture plate 110 includes a conical or tapered aperture 116 (shown inside a dotted circle for clarity) that extends through its center thickness and has a large inlet opening 116a in fluid communication with chamber 108 and a smaller exit opening 116b though which fluid droplets will be dispensed. Aperture plate 110 can be made of a flexible elastomer such as silicone rubber, VersaFlex, manufactured by VersaFlex Incorporated Kansas City, Kans. USA. Other elastomers with Young modulus of elasticity value between 0.5 GPa to 2 GPa can also be used. Dispensing system 104 further includes a stationary spherical member 114 that tangentially engages in pressure transmission relationship with the inlet opening 116a of the conical aperture providing hermetically sealed closure. In a preferred embodiment spherical member 114 is made of high density polyethylene (HDPE) that is harder than silicone thereby creating a tight closure as it engages with the softer aperture plate 110. Spherical member 114 preferably engages in pressure transmission relationship with the conical aperture 116a with a preload force of between 0.01N and 0.05N. The combination of aperture plate 110 and spherical member 114 provides a check valve as described above. Spherical member 114 is supported by pin member 112. Member 114 can have a shape other than spherical, since any shape capable of forming a good seal with aperture plate 110 can be used.

Aperture plate 110 can be retained to dispensing system 104 by a retaining ring 130, thereby creating a hermetically sealed closure.

Dispensing system 100 includes a venting tube 126 that is configured to equalize the pressure inside container 102 as the fluid is dispensed from the device. The opening 134 of venting tube 126 is extended above the fluid level 132 at any orientation that the device is held. Vent tube 126 can be connected via a 0.22 micron filter 128 to assure that the air that enters the device is sterile.

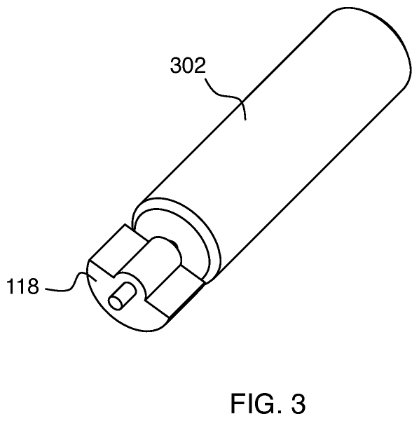

Dispensing system 100 further includes a vibration motor configured to oscillate chamber 108 and the fluid within the chamber. Here this motor is schematically shown as eccentric mechanical load 118 which vibrates the assembly as described when it is rotated by the motor (motor not shown in FIGS. 1A-B, but described below in connection with FIG. 3).

FIG. 1C is a 3D view of the embodiment of FIGS. 1A-B.

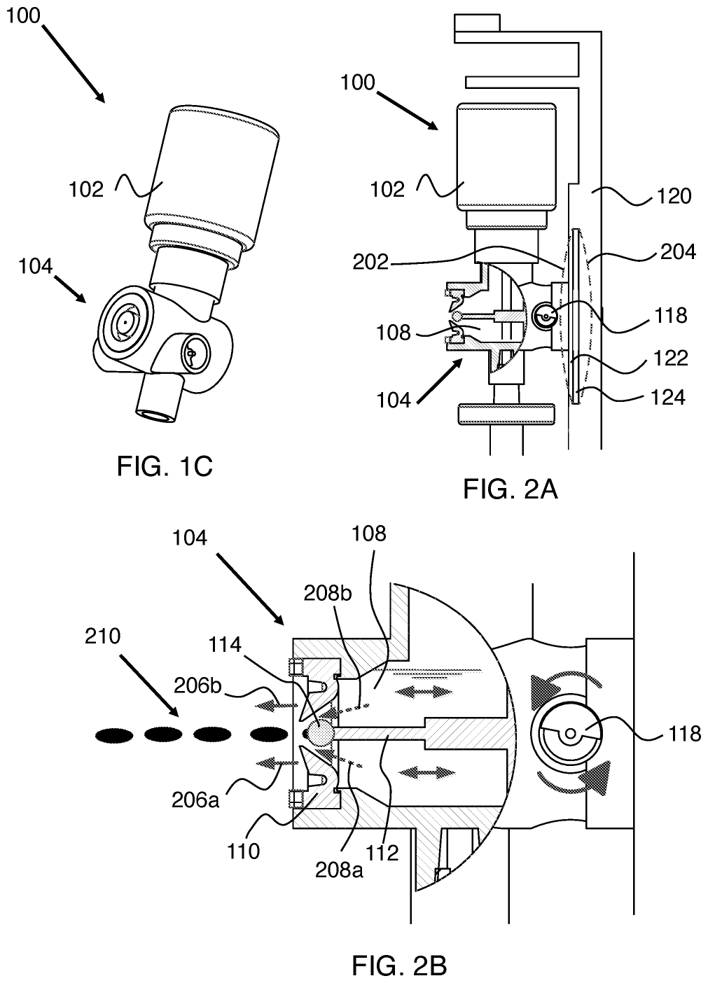

FIGS. 2A-B illustrate the response of the dispensing system 104 to oscillations. Vibrations of the motor induce oscillation to the body of dispensing system 104 which in turn generates fluid momentum within the chamber due to the dynamical interaction of the fluid with the solid structure of chamber 108. This phenomenon is often referred to as Solid-Fluid-Interaction (SFI). The momentum of the moving fluid exerts force that flexes the aperture plate 110 outwardly in the direction indicated by arrows 206a and 206b causing the aperture plate 110 to disengage from spherical member 114 thereby opening a fluid flow passage as indicated by the arrows 208a and 2086 and ejection of fluid droplets 210.

Dispensing device 100 is supported by a flexible beam 122 or other structural embodiments which allows it to oscillate freely, as schematically shown by motion excursions 202 and 204. Preferably the spring constant of the beam 122 is 0.05 N/mm to 0.5 N/mm. For example, beam 122 can be formed by fabricating a slot 124 in support structure 120 such that the resulting beam 122 has a thickness suitable for providing a spring constant as recited above.

FIG. 3 illustrates an exemplary vibrational motor 302. The DC Motor 302 of this example has a cylindrical body with a diameter of 4 mm and total length of 17 mm. An eccentric flywheel 118 is attached to the motor shaft. The flywheel has a mass of 1.7 grams with a center of mass about 0.7 mm from the center of rotation. The motor receives 4.5-12 VDC and rotates at 6000-12000 RPM generating centrifugal force of 0.3N at a rotation speed of 8000 RPM. Other DC motors that generate centrifugal force of 0.1-1N and rotation speed of 1000-50000 RPM can be used. The motor can be controlled by a timer circuit which is set to provide an ON time as required to deliver a dose of 8-12 micro-liter. The actuation ON time is 60-200 ms depending on the rheology of the fluid in use. A timer circuit which incorporates a 555 timer IC or a microprocessor-based timer with a 12 volt battery such as A23 alkaline battery may be used.

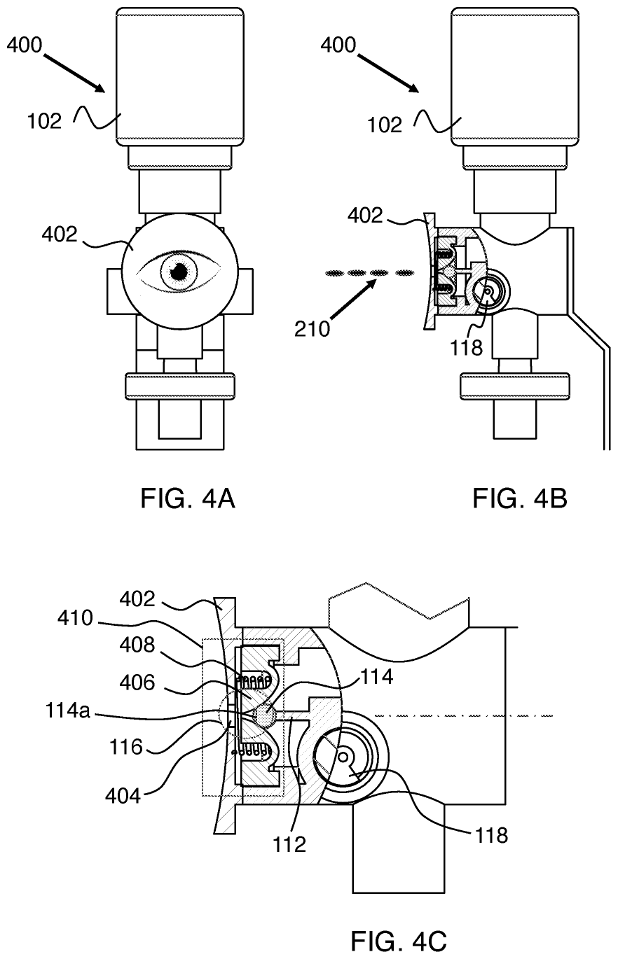

FIGS. 4A-C illustrate an alternative preferred embodiment of dispensing system 400. FIG. 4A illustrates a frontal view and FIG. 4B illustrates a side view of dispensing system 400. Dispensing system 400 includes a concave mirror 402 which assists in aligning device 400 and fluid stream 210 to the eye of the user. In use, dispensing device 400 is positioned in front of the eye such that the image of the eye appears sharply and in the center of mirror to the user. At this point the device is properly positioned in term of the distance of the eye from the nozzle 404 and its angular orientation relative to the eye. Upon actuation, stream 210 will be deposited precisely on the corneal surface of the eye. Device 400 preferably includes a 0.22 micron air filter configured to filter the vented air that flows into device 400, as in the previous example.

Device 400 includes a check valve having an aperture plate 406 with a conical aperture 116 that extends through its thickness. The check valve further includes a spherical member 114 that tangentially engages with the inlet opening of the conical aperture 116. In this example, the check valve also includes a compression spring 408 configured to force aperture plate 406 against spherical member 114. In this way a tight seal is created along the engagement line 114a, thus creating a tight and hermetic closure.

Spherical member 114 can be partially covered with an antimicrobial coating, specifically in the area of spherical member 114 that is not in contact with fluid in the chamber. The coated area thus extends between the engagement line 114a and the outlet of the conical aperture (i.e., to the left of 114a on FIG. 4C)). Examples of antimicrobial coatings include silver in metallic form, silver alloy or a non-metallic material that contains silver including salts of silver chloride and silver sulfadiazine. Other optional material includes biguanide derivatives, chlorohexidine diacetate and chlorohexidine digluconate.

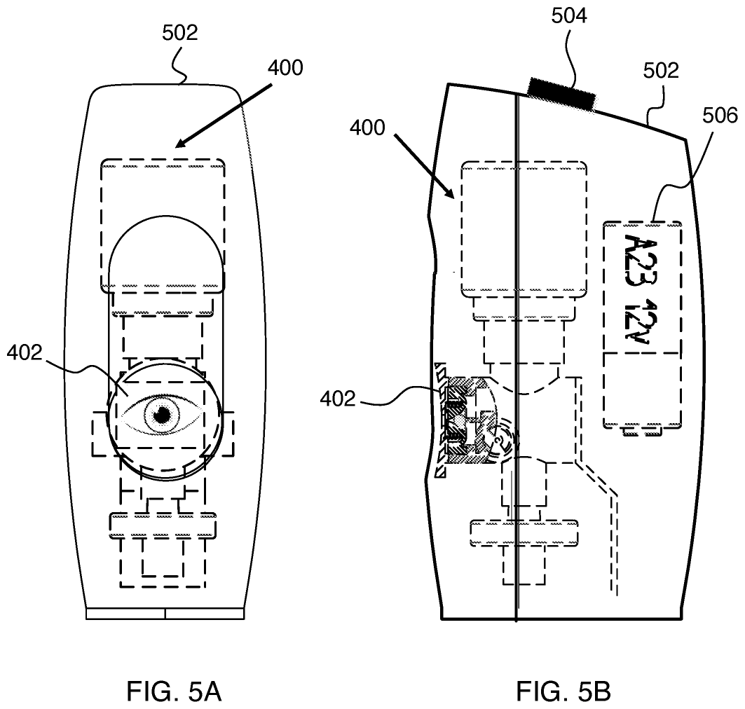

FIGS. 5A-B illustrate frontal and side views, respectively, of a dispensing device 400 packaged in a housing 502. Housing 502 provides a convenient enclosure to the dispensing system 400 that was described in relation in FIGS. 4A-C and is shown with dashed lines here. Housing 502 includes an electrical circuit (not shown) and a 12-volt battery (e.g., type A23) 506. The circuit controls the dispensing period such that a dose of 8-12 microliter is delivered to the ocular surface of the eye. Momentary switch 504 can be used to activate the device 400 such that a stream of droplets is ejected from the aperture as described earlier. FIGS. 5A-B also illustrate the mirror 402 which is visible on the front side of the device. Such a housing can also be used for a dispensing device 100 as described above.

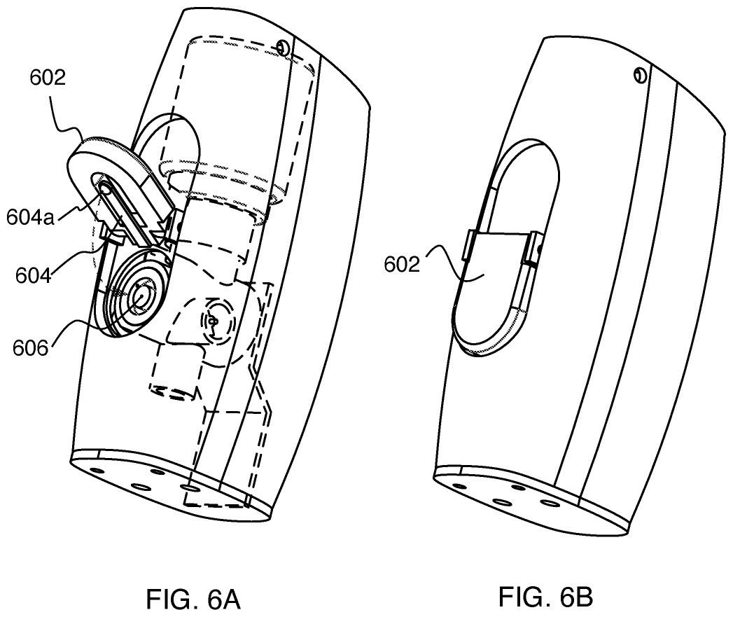

FIGS. 6A-B illustrate some preferred features of housings that can be used with embodiments such as device 100 and device 400 as described above. In this example, the housing includes a swivel cover 602 that covers dispensing nozzle 606 during periods of non-use. Swivel cover 602 provides a means to prevent bacterial contamination on the external areas where a residual fluid may be left following each use. Such residual fluid may contaminate subsequent stream as it is dispensed through the nozzle. Swivel cover 602 preferably includes a flexible member 604 that includes a surface 604a that is covered with antimicrobial coating. Surface 604a engages with the outlet opening of nozzle 606 when the swivel cover 602 is closed as illustrated in FIG. 6B. In this way antipathogen action is achieved. Alternatively, organic dyes with antiseptic action may also be used. For example, toluidine blue, methylene blue, gentian violet and acridine and related active substances such as acridine orange and acridine yellow as well as ethacridine lactate. Germicidal polymers such as polyhexanide are also possible. Materials that contain additives which contain metal-organic substances with an ionizing effect can also be used. Such additives are available from SteriOne GmbH Berlin, Germany. Examples of antimicrobial coatings that can be used on surface 604a include silver in metallic form, silver alloy or a non-metallic material that contains silver including salt of silver chloride and silver sulfadiazine. Other optional material includes biguanide derivatives, chlorohexidine diacetate and chlorohexidine digluconate.

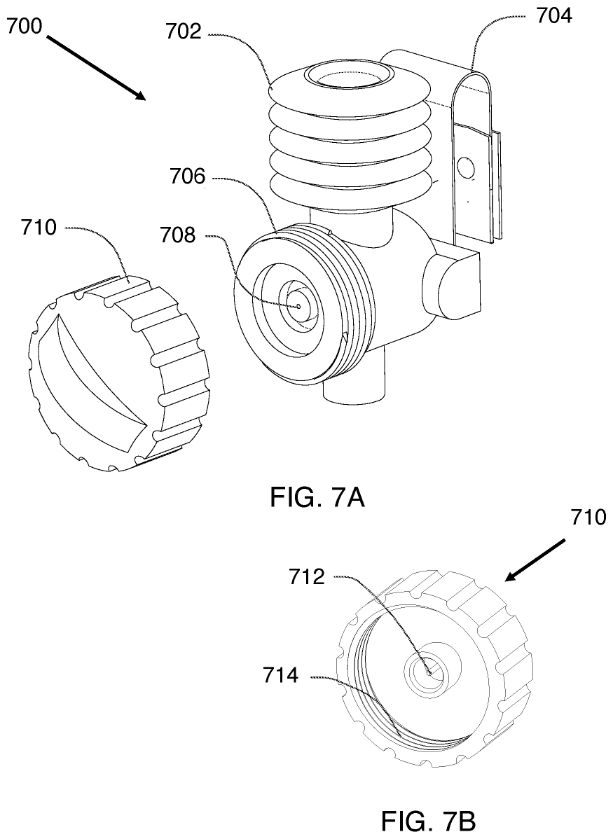

FIGS. 7A-B illustrate a dispensing system 700 utilizing an alternative fluid reservoir 702 configured to eliminate the need for venting and air filtering as previously described in relation to other embodiments. Dispensing system 700 includes a collapsible fluid chamber (e.g., a bellows) 702 containing ophthalmic solution to be dispensed. When fluid is dispensed the internal pressure inside the bellows 702 is gradually reduced which results in axial deformation of bellows 702 which in turn equalizes the pressure inside bellows 702 with the atmospheric pressure. In this way fluid inside the bellows 702 does not need to be vented to the atmosphere and further may not have to be in contact with air and particularly oxygen. Such a system is particularly advantageous for ophthalmic fluids that are sensitive to oxidation. Dispensing system 700 further includes cap 710 that seals off the dispensing nozzle during non-use period. FIG. 7B illustrates a frontal view of cap 710. It can be seen that cap 710 has an internal thread 714 that engages with the external thread 706. Cap 710 further includes a central pin 712 that is configured to seal off dispensing aperture 708 during periods of non-use. Apparatus 700 includes a flexible mount 704 to permit a vibration motor to vibrate the apparatus to eject liquid as described above.

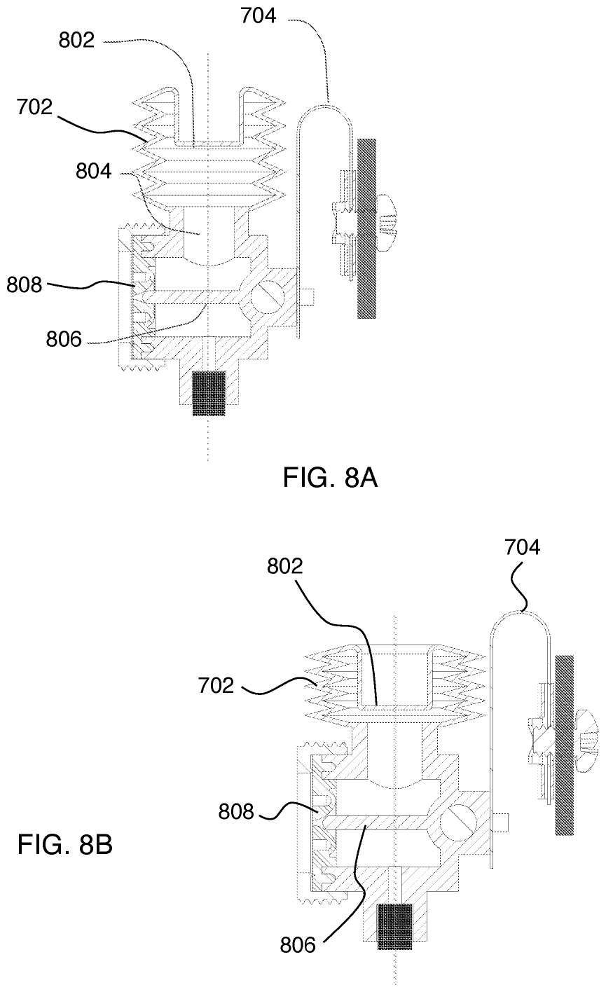

FIG. 8A illustrates a cross-sectional view of the dispensing system of this example when bellows 702 is filled with fluid while FIG. 8B shows the bellows 702 in a collapsed or nearly empty state. It can be seen that the open end of bellows 702 is connected to the dispensing chamber 804. Preferably the sealed end of the bellows is recessed inwardly toward the bellows forming a cup-shaped indentation 802 as shown. In this way the unused volume that is left in bellows 702 once it is fully collapsed is minimized, as shown on FIG. 8B. Here stationary member 806 and flexible aperture plate 808 form a check value that operates as described above when the apparatus is vibrated by a vibration motor.

What is claimed is:

1. A method for delivering fluid to a targeted site, the method comprising:
using an actuator to oscillate a fluid containing chamber in fluid communication with a reservoir to generate fluid momentum in the chamber;
allowing a pin positioned within the chamber and in fluid-tight engagement with an aperture of the chamber to disengage from one another in the presence of the generated fluid momentum so that the fluid can be ejected through the aperture to the targeted site; and
collapsing the reservoir as the fluid is ejected to equalize pressure within the reservoir with atmospheric pressure.

2. The method of claim 1, wherein the fluid is an ophthalmic fluid sensitive to oxidation.

3. The method of claim 1, wherein, in the using step, the reservoir is a bellows.

4. The method of claim 3, wherein, in the collapsing step, the bellows is axially deformed to equalize the pressure therein with the atmospheric pressure outside the bellows.

5. The method of claim 1, wherein, in the collapsing step, the pressure within the reservoir is equalized without introducing air into the reservoir.

6. The method of claim 1, wherein, in the collapsing step, the reservoir is attached to the chamber at a first end and a second end, opposite the first end, is recessed inward as the reservoir is collapsed.

7. The method of claim 6, wherein the second end forms a cup-shaped indentation.

8. The method of claim 1, wherein, in the allowing step, the aperture is a flexible aperture.

9. The method of claim 1, further comprising:
reengaging the aperture with the pin to seal the aperture after the fluid is delivered.

10. The method of claim 9, wherein the reengaging step further comprises preventing an ingress of bacteria or contaminants as a result of an engagement between the aperture and the pin.

11. The method of claim 1, further comprising:
biasing the aperture against the pin via a compression spring.

12. An apparatus for delivering a fluid to a targeted site, the apparatus comprising:
a fluid containing chamber having an aperture through which the fluid can be selectively dispensed;
an actuator engaging the chamber to generate fluid momentum therein to open the aperture for fluid ejection therethrough; and
a reservoir in fluid communication with the chamber and collapsible to equalize pressure within the reservoir with atmospheric pressure as the fluid is being ejected.

13. The apparatus of claim 12, wherein the fluid is an ophthalmic fluid sensitive to oxidation.

14. The apparatus of claim 12, wherein the aperture is flexible.

15. The apparatus of claim 12, wherein the reservoir is a bellows.

16. The apparatus of claim 15, wherein the bellows is axially deformed to equalize the pressure therein with the atmosphere.

17. The apparatus of claim 12, wherein the pressure within the reservoir is equalized without introducing air into the reservoir.

18. The apparatus of claim 12, wherein the reservoir is attached to the chamber at a first end and a second end, opposite the first end, is recessed inward as the reservoir is collapsed to minimize unused fluid within the reservoir.

19. The apparatus of claim 18, wherein the second end forms a cup-shaped indentation.

20. The apparatus of claim 13, further comprising a pin member engaging the aperture such that the aperture, as a result of the fluid momentum, is disengaged from the pin member.
